(19) United States
(12) Patent Application Publication
(10) Pub. No.: US 2026/0011400 A1
(43) Pub. Date: Jan. 8, 2026

(54) MACHINE LEARNING SYSTEM AND METHOD FOR PREDICTING BLOOD BRAIN BARRIER PERMEABILITY

(71) Applicant: Lantern Pharma Inc., Dallas, TX (US)

(72) Inventors: Umesh Kathad, Dallas, TX (US); Joseph McDermott, Atlanta, GA (US); Richard Fontenot, Dallas, TX (US); Panna Sharma, Atlanta, GA (US)

(73) Assignee: Lantern Pharma Inc., Dallas, TX (US)

(21) Appl. No.: 19/328,348

(22) Filed: Sep. 15, 2025

Related U.S. Application Data
(63) Continuation of application No. PCT/US2024/019851, filed on Mar. 14, 2024.
(60) Provisional application No. 63/452,108, filed on Mar. 14, 2023.

(51) Int. Cl. G16B 15/30; G16B 40/20

(52) U.S. Cl. CPC G16B 15/30; G16B 40/20

(57) ABSTRACT

A machine learning system and method for predicting blood-brain barrier permeability is provided. The system obtains samples of data associated with molecules from various data sources, converts the samples into structural representations, and generates a plurality of features from the structural representations, such as fingerprint representations. Tests are executed on the features to determine blood-brain barrier permeability dependency, The system analyzes the ratio of permeable to non-permeable samples in the samples of data and augments the samples with synthetic data to create a balanced dataset if an imbalance between the types of samples is detected. The system reduces the features utilized for training the machine learning utilizing a technique, such as logistic regression, to create a selected set of features for the balanced dataset. The system trains a machine learning model using the balanced dataset and utilizing the machine learning model to predict blood-brain barrier permeability for the candidate molecule.

### CROSS-REFERENCE TO RELATED APPLICATIONS

This is application a continuation of International Patent Application No PCT/US2024/019851, filed on Mar. 14, 2024, which claims priority to and the benefit of U.S. Provisional Patent Application Ser. No. 63/452,108, filed on Mar. 14, 2023, each of which is hereby incorporate by reference in its entirety.

### FIELD OF THE INVENTION

The present application relates to artificial intelligence technologies, machine learning technologies, blood brain barrier permeability prediction technologies, molecule design technologies, data analysis technologies, and, more particularly, to machine learning system and accompanying methods for predicting blood brain barrier permeability.

### BACKGROUND

The blood-brain barrier is a semipermeable membrane that effectively separates circulating blood from the extracellular brain fluid in a person's central nervous system. Blood-brain barrier permeability is the ability of various substances to cross through the barrier between the person's bloodstream and brain tissue. The various cells in the blood-brain barrier prevent the passage of many types of molecules, such as those that are harmful to the brain. However, the blood-brain barrier does enable certain substances, such as water, oxygen, lipid-soluble molecules, to cross to allow essential nutrients to pass through. Currently, being able to effectively enhance blood-brain barrier permeability for drug delivery purposes is a much sought after goal. To that end, various drug companies have employed the use of technological tools, such as software and artificial intelligence systems to determine or predict the bloodbrain barrier permeability of a particular molecule under consideration for a drug.

Although blood-brain barrier permeability prediction with certain existing machine learning and deep learning methods based on molecular structure have been shown to be somewhat accurate, current approaches suffer from several major defects that reduce their applicability and usefulness. For example, current state-of-the-art methods produce black box models that cannot provide insight as to why a molecule is predicted to be permeable or impermeable, thereby making it nearly impossible to use molecular predictions as a tool to improve blood-brain barrier permeability. Based on at least the foregoing, there remains room for substantial enhancements to existing technologies and processes and for the development of new technologies and processes that provide blood-brain barrier permeability predictive capabilities. For example, current technologies may be improved and enhanced so as to provide for improved artificial intelligence model performance on test data, more efficient use of computing resources while generating models and predictions, greater interpretative capabilities, and various other benefits. Such enhancements and improvements to methodologies and technologies may provide for greater understanding of which portions of a molecule correlate with blood-brain barrier permeability and ultimately which molecules are optimal candidates for treating various health conditions.

### SUMMARY

A system and accompanying methods for predicting blood-brain barrier permeability are disclosed. In particular, the system and methods involve utilizing unique processes to generate machine learning models that are capable of effectively predicting whether a particular molecule under consideration has blood-brain barrier permeability, while simultaneously utilizing fewer computing resources and features. As a result, the machine learning models generated by utilizing the system and methods are more robust and interpretable. The functionality provided by the system and methods also facilitate the understanding of how specific chemical structures of a molecule under consideration impact blood-brain barrier permeability, and how molecule design can be improved or modified to enhance blood-brain barrier permeability. Still further, the system and methods provide unique model interpretation analysis that advance chemical engineering of blood-brain barrier permeable therapeutics.

In certain embodiments, a system for predicting blood brain barrier permeability is provided. In certain embodiments, the system can include a memory that stores instructions and a processor that is configured to execute the instructions to configure the processor to perform various operations. In certain embodiments, the processor can be configured to generate a plurality of features for one or more structural representations of one or more molecules. In certain embodiments, the plurality of features can include one or more molecular fingerprint representations associated with the one or more molecules, descriptors, graph embeddings, any other features, or a combination thereof. In certain embodiments, the processor can be configured to execute a chi-square test on the plurality of features of the one or more structural representations to determine whether blood-brain barrier permeability is dependent on the one or more molecular fingerprint representations. In certain embodiments, the processor can be configured to determine a ratio of permeable samples and non-permeable samples associated with the one or more molecules and containing the one or more molecular fingerprint representations. In certain embodiments, the processor can be configured to augment, based on the ratio, a training dataset comprising the permeable samples and non-permeable samples by utilizing a k-nearest neighbor algorithm to create synthetic data for a minority class of the permeable and non-permeable samples until sample counts for the training dataset are balanced between the permeable samples and non-permeable samples to generate a balanced training dataset. In certain embodiments, the processor can be configured to reduce the plurality of features utilized for the balanced training dataset using a logistic regression with least absolute shrinkage to create a selected set of features for the balanced training dataset. In certain embodiments, the processor can be configured to train, by utilizing the balanced training dataset with the selected set of features, an ensemble meta learner to predict bloodbrain barrier permeability. In certain embodiments, the processor can be configured to analyze, by utilizing the ensemble meta learner, a candidate molecule for blood-brain barrier permeability. In certain embodiments, the processor can be configured to generate, by utilizing the ensemble meta learner, a prediction of whether the candidate molecule has blood-brain barrier permeability.

In certain embodiments, a method for blood-brain barrier permeability is disclosed. The method may include a memory that stores instructions and a processor that executes the instructions to perform the functionality of the method. In particular, the method may include generating a plurality of features for one or more structural representations of one or more molecules. In certain embodiments, the plurality of features can include one or more molecular fingerprint representations associated with the one or more molecules. In certain embodiments, the method can include executing a chi-square test on the plurality of features of the one or more structural representations to determine whether blood-brain barrier permeability is dependent on the one or more molecular fingerprint representations. In certain embodiments, the method can include determining a ratio of permeable samples and non-permeable samples associated with the one or more molecules and containing the one or more molecular fingerprint representations. In certain embodiments, the method can include augmenting, based on the ratio, a training dataset comprising the permeable samples and non-permeable samples by utilizing a k-nearest neighbor algorithm to create synthetic data for a minority class of the permeable and non-permeable samples until sample counts for the training dataset are balanced between the permeable samples and non-permeable samples to generate a balanced training dataset. In certain embodiments, the method can include reducing the plurality of features utilized for the balanced training dataset using a logistic regression with least absolute shrinkage to create a selected set of features for the balanced training dataset. In certain embodiments, the method can include training, by utilizing the balanced training dataset with the selected set of features, an ensemble meta learner to predict blood-brain barrier permeability. In certain embodiments, the method can include analyzing, by utilizing the ensemble meta learner, a candidate molecule for blood-brain barrier permeability. In certain embodiments, the method can include generating, by utilizing the ensemble meta learner, a prediction of whether the candidate molecule has blood-brain barrier permeability. The method can include and/or be modified to include any of the functionality of the system and/or any of the functionality described in the present disclosure.

According to further embodiments, a computer-readable device comprising instructions, which, when loaded and executed by a processor cause the processor to perform operations, the operations comprising: generate a plurality of features for at least one structural representation of at least one molecule, wherein the plurality of features comprise at least one molecular fingerprint representation associated with the at least one molecule; execute a chi-square test on the plurality of features of the at least one structural representation to determine whether blood-brain barrier permeability is dependent on the at least one molecular fingerprint representation; determine a ratio of permeable samples and non-permeable samples associated with the at least one molecule and containing the at least one molecular fingerprint representation; augment, based on the ratio, a training dataset comprising the permeable samples and non-permeable samples by utilizing a k-nearest neighbor algorithm to create synthetic data for a minority class of the permeable and non-permeable samples until sample counts for the training dataset are balanced between the permeable samples and non-permeable samples to generate a balanced training dataset; reduce the plurality of features utilized for the balanced training dataset using a logistic regression with least absolute shrinkage to create a selected set of features for the balanced training dataset; train, by utilizing the balanced training dataset with the selected set of features, an ensemble meta learner to predict blood-brain barrier permeability; analyze, by utilizing the ensemble meta learner, a candidate molecule for bloodbrain barrier permeability; and generate, by utilizing the ensemble meta learner, a prediction of whether the candidate molecule has blood-brain barrier permeability.

These and other features of the systems and methods for predicting blood-brain barrier permeability are described in the following detailed description, drawings, and appended claims.

### BRIEF DESCRIPTION OF THE DRAWINGS

FIG. 1 is a schematic diagram of a system for predicting blood-brain barrier permeability according to embodiments of the present disclosure.

FIG. 2 illustrates a schematic diagram featuring a system for building a machine learning model for predicting blood-brain barrier permeability according to embodiments of the present disclosure.

FIG. 3 illustrates a table illustrating exemplary feature generation methods for generating features to be considered for building a machine learning model for predicting blood-brain barrier permeability according to embodiments of the present disclosure.

FIG. 4 illustrates a table featuring a reduced set of features to be utilized to building a machine learning model for predicting blood-brain barrier permeability according to embodiments of the present disclosure.

FIG. 5 illustrates an exemplary process flow diagram for generating features, reducing features, training a machine learning model, and analyzing a candidate molecule for blood-brain barrier permeability according to embodiments of the present disclosure.

FIG. 6 illustrates a table illustrating an exemplary mapping of 2D autocorrelation features back to atomic properties according to embodiments of the present disclosure.

FIG. 7 illustrates a mapping of fingerprints to a portion of a molecule according to embodiments of the present disclosure.

FIG. 8 is a flow diagram illustrating a sample method for building a machine learning model for predicting blood-brain barrier permeability and utilizing the machine learning model to predict permeability for a molecule under consideration according to embodiments of the present disclosure.

FIG. 9 is a schematic diagram of a machine in the form of a computer system within which a set of instructions, when executed, may cause the machine to facilitate predictions for blood-brain permeability for molecules according to embodiments of the present disclosure.

### DETAILED DESCRIPTION OF THE DRAWINGS

A system 100 and accompanying methods for predicting blood-brain barrier permeability are disclosed. In particular, the system 100 and methods involve utilizing novel processes to generate machine learning models that are capable of effectively predicting the blood-brain barrier permeability of a particular molecule under consideration, while utilizing fewer computing resources and features than existing technologies. The functionality provided by the system 100 and methods also generate information that indicates how specific chemical structures of a molecule under consideration impact blood-brain barrier permeability, and how molecule design can be improved or modified to enhance blood-brain barrier permeability. Furthermore, the system 100 and methods provide unique model interpretation analysis that advance chemical engineering of blood-brain barrier permeable therapeutics.

In certain embodiments, a system for predicting blood brain barrier permeability is provided. In certain embodiments, the system can include a memory that stores instructions and a processor that is configured to execute the instructions to configure the processor to perform various operations. In certain embodiments, the processor can be configured to generate a plurality of features for one or more structural representations of one or more molecules. In certain embodiments, the plurality of features can include one or more molecular fingerprint representations associated with the one or more molecules. In certain embodiments, the processor can be configured to execute a chi-square test on the plurality of features of the one or more structural representations to determine whether blood-brain barrier permeability is dependent on the one or more molecular fingerprint representations. In certain embodiments, the processor can be configured to determine a ratio of permeable samples and non-permeable samples associated with the one or more molecules and containing the one or more molecular fingerprint representations. In certain embodiments, the processor can be configured to augment, based on the ratio, a training dataset comprising the permeable samples and non-permeable samples by utilizing a k-nearest neighbor algorithm to create synthetic data for a minority class of the permeable and non-permeable samples until sample counts for the training dataset are balanced between the permeable samples and non-permeable samples to generate a balanced training dataset. In certain embodiments, the processor can be configured to reduce the plurality of features utilized for the balanced training dataset using a logistic regression with least absolute shrinkage to create a selected set of features for the balanced training dataset. In certain embodiments, the processor can be configured to train, by utilizing the balanced training dataset with the selected set of features, an ensemble meta learner to predict bloodbrain barrier permeability. In certain embodiments, the processor can be configured to analyze, by utilizing the ensemble meta learner, a candidate molecule for blood-brain barrier permeability. In certain embodiments, the processor can be configured to generate, by utilizing the ensemble meta learner, a prediction of whether the candidate molecule has blood-brain barrier permeability.

In certain embodiments, the processor can be configured to generate the one or more structural representations of the one or more molecules by translating a three-dimensional structure of the one or more molecules into a string of symbols discernible by the system. In certain embodiments, the processor can be configured to determine that blood-brain barrier permeability is dependent on the one or more molecular fingerprint representation based on the one or more molecular fingerprints having a p-value of less than 0.05 (or other desired value).

In certain embodiments, the processor can be configured to classify the permeable samples of the plurality of samples as permeable based on fingerprints (i.e., fingerprint representations) associated with the permeable samples having a threshold blood-brain permeability. In certain embodiments, the processor can be further configured to classify the non-permeable samples of the plurality of samples as non-permeable based on fingerprints associated with the non-permeable samples having less than the threshold blood-brain permeability value. In certain embodiments, the processor can be configured to determine that the non-permeable samples are the minority class in the plurality of samples based on the permeable samples being greater in number than the non-permeable samples.

In certain embodiments, the processor can be configured to reduce, using the logistic regression, coefficients of features of the plurality of features to zero to eliminate the features from being included in the selected set of features used for training a machine learning model to generate predictions regarding blood-brain barrier permeability or other predictions. In certain embodiments, the processor can be configured to rank features in the selected set of features in order of importance based on an absolute value of each coefficient of the features in the selected set of features. In certain embodiments, the processor can be configured to generate an ensemble meta learner from one or more base learner models that are trained based on the balanced training dataset and by utilizing a logistic regression, a deep neural network, or a combination thereof. In certain embodiments, the processor can be configured to determine a predicted probability of permeability for holdout validation samples not included in the balanced training dataset. In certain embodiments, the processor can be configured to utilize the predicted probability of permeability for the holdout validation samples as an input to a logistic regression meta-learner ensemble model. In certain embodiments, the processor can be configured to select the ensemble meta learner as a combination of base learner models having a highest area under a receiver operating characteristic curve.

In certain embodiments, a method for blood-brain barrier permeability is disclosed. The method may include a memory that stores instructions and a processor that executes the instructions to perform the functionality of the method. In particular, the method may include generating a plurality of features for one or more structural representations of one or more molecules. In certain embodiments, the plurality of features can include one or more molecular fingerprint representations associated with the one or more molecules. In certain embodiments, the method can include executing a chi-square test on the plurality of features of the one or more structural representations to determine whether blood-brain barrier permeability is dependent on the one or more molecular fingerprint representations. In certain embodiments, the method can include determining a ratio of permeable samples and non-permeable samples associated with the one or more molecules and containing the one or more molecular fingerprint representations. In certain embodiments, the method can include augmenting, based on the ratio, a training dataset comprising the permeable samples and non-permeable samples by utilizing a k-nearest neighbor algorithm to create synthetic data for a minority class of the permeable and non-permeable samples until sample counts for the training dataset are balanced between the permeable samples and non-permeable samples to generate a balanced training dataset. In certain embodiments, the method can include reducing the plurality of features utilized for the balanced training dataset using a logistic regression with least absolute shrinkage to create a selected set of features for the balanced training dataset. In certain embodiments, the method can include training, by utilizing the balanced training dataset with the selected set of features, an ensemble meta learner to predict blood-brain barrier permeability. In certain embodiments, the method can include analyzing, by utilizing the ensemble meta learner, a candidate molecule for blood-brain barrier permeability. In certain embodiments, the method can include generating, by utilizing the ensemble meta learner, a prediction of whether the candidate molecule has blood-brain barrier permeability.

In certain embodiments, the method can include identifying, by utilizing the ensemble meta learner, a specific portion of the candidate molecule that has the blood-barrier permeability. In certain embodiments, the method can include generating the ensemble meta learner from at least one base learner model that is trained based on the balanced training dataset and by utilizing the logistic regression, a deep neural network, or a combination thereof. In certain embodiments, the method can include stopping the training of one or more bases learner model utilized to generate the ensemble meta learner at an epoch representing a highest area under a receiver operating characteristic curve on holdout samples. In certain embodiments, the method can include reducing, using the logistic regression, coefficients of features of the plurality of features to zero to eliminate the features from being included in the selected set of features. In certain embodiments, the method can include generating the one or more structural representations of the one or more molecules by translating a three-dimensional structure of the one or more molecules into a string of symbols. In certain embodiments, the method can include determining a correlation of blood-brain permeability between the at least one molecule and the at least one candidate molecule.

According to further embodiments, a computer-readable device comprising instructions, which, when loaded and executed by a processor cause the processor to perform operations, the operations comprising: generate a plurality of features for at least one structural representation of at least one molecule, wherein the plurality of features comprise at least one molecular fingerprint representation associated with the at least one molecule; execute a chi-square test on the plurality of features of the at least one structural representation to determine whether blood-brain barrier permeability is dependent on the at least one molecular fingerprint representation; determine a ratio of permeable samples and non-permeable samples associated with the at least one molecule and containing the at least one molecular fingerprint representation; augment, based on the ratio, a training dataset comprising the permeable samples and non-permeable samples by utilizing a k-nearest neighbor algorithm to create synthetic data for a minority class of the permeable and non-permeable samples until sample counts for the training dataset are balanced between the permeable samples and non-permeable samples to generate a balanced training dataset; reduce the plurality of features utilized for the balanced training dataset using a logistic regression with least absolute shrinkage to create a selected set of features for the balanced training dataset; train, by utilizing the balanced training dataset with the selected set of features, an ensemble meta learner to predict blood-brain barrier permeability; analyze, by utilizing the ensemble meta learner, a candidate molecule for bloodbrain barrier permeability; and generate, by utilizing the ensemble meta learner, a prediction of whether the candidate molecule has blood-brain barrier permeability.

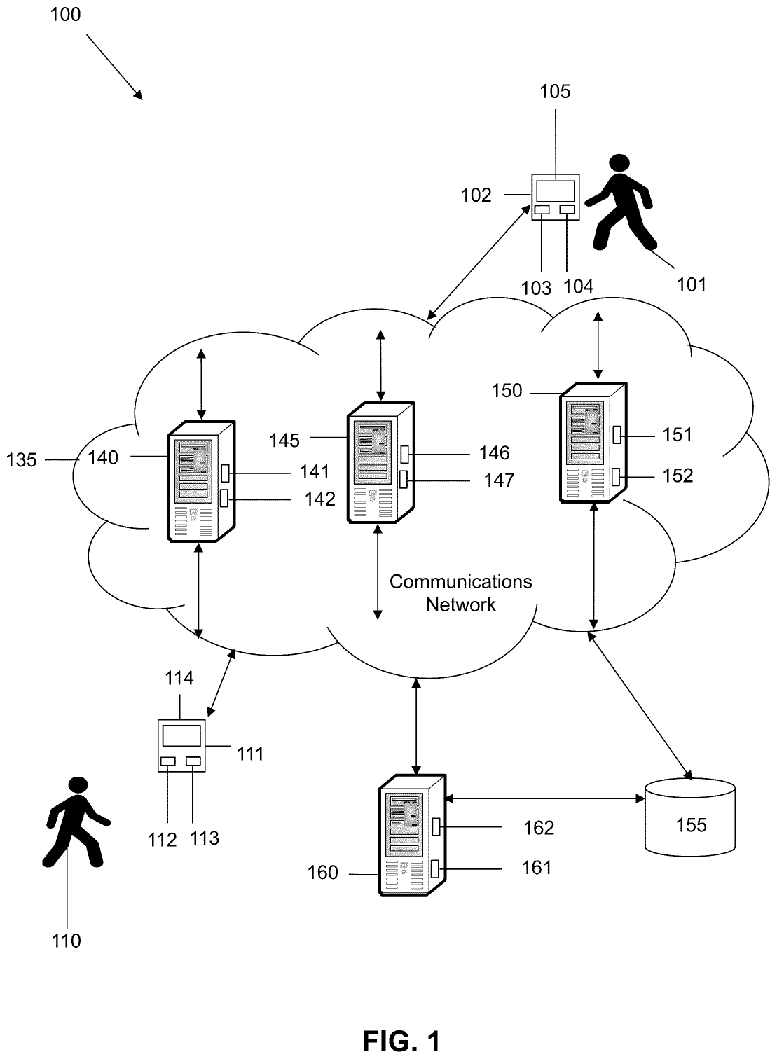

As shown in FIG. 1, a system for predicting blood-brain barrier permeability according to embodiments of the present disclosure is disclosed. Notably, the system 100 may be configured to support, but is not limited to supporting, automation systems, blood-brain barrier prediction systems, data analytics systems and services, data collation and processing systems and services, artificial intelligence services and systems, machine learning services and systems, content delivery services, cloud computing services, satellite services, telephone services, voice-over-internet protocol services (VOIP), software as a service (SaaS) applications, platform as a service (PaaS) applications, social media applications and services, operations management applications and services, productivity applications and services, mobile applications and services, and/or any other computing applications and services. Notably, the system 100 may include a first user 101, who may utilize a first user device 102 to access data, content, and services, or to perform a variety of other tasks and functions. As an example, the first user 101 may utilize first user device 102 to transmit signals to access various online services and content, such as those available on an internet, on other devices, and/or on various computing systems. As another example, the first user device 102 may be utilized by the first user 101 to access an application, devices, and/or components of the system 100 that provide any or all of the operative functions of the system 100. For example, the first user 101 may utilize the first user device 102 to access an application supported by machine learning models that is utilized to determine whether a particular molecule (e.g., a molecule for a drug) under consideration or evaluation has blood-brain barrier permeability. In certain embodiments, the first user 101 may be any type of person, a robot, a humanoid, a program, a computer, any type of user, or a combination thereof, that may be located in a particular environment.

In certain embodiments, the first user 101 may be a person that may be seeking to determine whether a particular molecule of interest has blood-brain barrier permeability. In certain embodiments, the first user device 102 may be utilized by the first user to interact with the system 100, other users of the system 100, or a combination thereof. In certain embodiments, the first user device 102 may include a memory 103 that includes instructions, and a processor 104 that executes the instructions from the memory 103 to perform the various operations that are performed by the first user device 102. In certain embodiments, the processor 104 may be hardware, software, or a combination thereof. The first user device 102 may also include an interface 105 (e.g. screen, monitor, graphical user interface, etc.) that may enable the first user 101 to interact with various applications executing on the first user device 102 and to interact with the system 100. In certain embodiments, the first user device 102 may be and/or may include a computer, any type of sensor, a laptop, a set-top-box, a tablet device, a phablet, a server, a mobile device, a smartphone, a smart watch, and/or any other type of computing device. Illustratively, the first user device 102 is shown as a smartphone device in FIG. 1. In certain embodiments, the first user device 102 may be utilized by the first user 101 to control and/or provide some or all of the operative functionality of the system 100.

In addition to using first user device 102, the first user 101 may also utilize and/or have access to additional user devices. As with first user device 102, the first user 101 may utilize the additional user devices to transmit signals to access various online services and content. The additional user devices may include memories that include instructions, and processors that executes the instructions from the memories to perform the various operations that are performed by the additional user devices. In certain embodiments, the processors of the additional user devices may be hardware, software, or a combination thereof. The additional user devices may also include interfaces that may enable the first user 101 to interact with various applications executing on the additional user devices and to interact with the system 100. In certain embodiments, the first user device 102 and/or the additional user devices may be and/or may include a computer, any type of sensor, a laptop, a set-top-box, a tablet device, a phablet, a server, a mobile device, a smartphone, a smart watch, and/or any other type of computing device, and/or any combination thereof. Sensors may include, but are not limited to, cameras, motion sensors, acoustic/audio sensors, pressure sensors, temperature sensors, light sensors, heart-rate sensors, blood pressure sensors, sweat detection sensors, eyetracking sensors, breath-detection sensors, stress-detection sensors, any type of health sensor, humidity sensors, any type of sensors, or a combination thereof.

The first user device 102 and/or additional user devices may belong to and/or form a communications network. In certain embodiments, the communications network may be a local, mesh, or other network that enables and/or facilitates various aspects of the functionality of the system 100. In certain embodiments, the communications network may be formed between the first user device 102 and additional user devices through the use of any type of wireless or other protocol and/or technology. For example, user devices may communicate with one another in the communications network by utilizing any protocol and/or wireless technology, satellite, fiber, or any combination thereof. Notably, the communications network may be configured to communicatively link with and/or communicate with any other network of the system 100 and/or outside the system 100.

In certain embodiments, the first user device 102 and additional user devices belonging to the communications network may share and exchange data with each other via the communications network. For example, the user devices may share information associated with a molecule (e.g., a molecule under consideration or evaluation by the system 100) with each other, information relating to the chemical structure of the molecule, information relating to whether the molecule has blood-brain barrier permeability, information relating to machine learning models that predict blood-brain barrier permeability for molecules, information relating to fingerprints generated for a molecule, information relating to feature generated from structural and/or fingerprint representations of a molecule, information relating to features selected to train machine learning models for generating the predictions, information relating to the various components of the user devices, information associated with images and/or content accessed by a user of the user devices, information identifying the locations of the user devices, information indicating the types of sensors that are contained in and/or on the user devices, information identifying the applications being utilized on the user devices, information identifying how the user devices are being utilized by a user, information identifying user profiles for users of the user devices, information identifying device profiles for the user devices, information identifying the number of devices in the communications network, information identifying devices being added to or removed from the communications network, any other information, or any combination thereof.

In addition to the first user 101, the system 100 may also include a second user 110. In certain embodiments, the second user 110 may seek to determine whether a different molecule has blood-brain barrier permeability. In certain embodiments, the second user 110 can be a patient or other user that may be a subject for administration of a candidate molecule to determine blood-brain barrier permeability of the candidate molecule. In certain embodiments, the second user device 111 may be utilized by the second user 110 to transmit signals to request various types of content, services, and data provided by and/or accessible by communications network 135 or any other network in the system 100, such as, but not limited to, artificial intelligence and/or machine learning models of the system 100. In certain embodiments, the second user device 111 may be utilized by the second user 110 to perform any operative functionality of the system 100, or a combination thereof. In further embodiments, the second user 110 may be a robot, a computer, a vehicle, a humanoid, an animal, any type of user, or any combination thereof. The second user device 111 may include a memory 112 that includes instructions, and a processor 113 that executes the instructions from the memory 112 to perform the various operations that are performed by the second user device 111. In certain embodiments, the processor 113 may be hardware, software, or a combination thereof. The second user device 111 may also include an interface 114 (e.g. screen, monitor, graphical user interface, etc.) that may enable the first user 101 to interact with various applications executing on the second user device 111 and, in certain embodiments, to interact with the system 100. In certain embodiments, the second user device 111 may be a computer, a laptop, a set-top-box, a tablet device, a phablet, a server, a mobile device, a smartphone, a smart watch, and/or any other type of computing device. Illustratively, the second user device 111 is shown as a mobile device in FIG. 1. In certain embodiments, the second user device 111 may also include sensors, such as, but are not limited to, cameras, audio sensors, motion sensors, pressure sensors, temperature sensors, light sensors, heart-rate sensors, blood pressure sensors, sweat detection sensors, breath-detection sensors, eye-tracking sensors, stress-detection sensors, any type of health sensor, humidity sensors, any type of sensors, or a combination thereof.

In certain embodiments, the first user device 102, the additional user devices, and/or the second user device 111 may have any number of software applications and/or application services stored and/or accessible thereon. For example, the first user device 102, the additional user devices, and/or the second user device 111 may include applications for controlling and/or accessing the operative features and functionality of the system 100, applications for controlling and/or accessing any device of the system 100, applications for generating fingerprint representations of molecules, applications for generating machine learning models, applications for generating features from representations of molecules, applications for augmenting a sample set containing sample imbalances, applications for generating predictions for blood-brain barrier permeability, cloud-based applications, VOIP applications, other types of phone-based applications, product-ordering applications, business applications, e-commerce applications, media streaming applications, content-based applications, media-editing applications, database applications, gaming applications, internet-based applications, browser applications, mobile applications, service-based applications, productivity applications, video applications, music applications, social media applications, any other type of applications, any types of application services, or a combination thereof. In certain embodiments, the software applications may support the functionality provided by the system 100 and methods described in the present disclosure. In certain embodiments, the software applications and services may include one or more graphical user interfaces so as to enable the first and/or potentially second users 101, 110 to readily interact with the software applications. The software applications and services may also be utilized by the first and/or potentially second users 101, 110 to interact with any device in the system 100, any network in the system 100, or any combination thereof. In certain embodiments, the first user device 102, the additional user devices, and/or potentially the second user device 111 may include associated telephone numbers, device identities, or any other identifiers to uniquely identify the first user device 102, the additional user devices, and/or the second user device 111.

In certain embodiments, for example, the first user 101 may utilize the first user device 102 to initiate operation of the system 100 itself. For example, the first user 101 can initiate one or more applications supporting the functionality of the system 100 and can activate operation of one or more machine learning models to generate predictions regarding bloodbrain barrier permeability of any number of molecules under consideration. In certain embodiments, the first user 101 can utilize a user interface of the first user device 102 to interact with the application and can trigger training of a machine learning model, trigger generation of features from samples (e.g., labeled or unlabeled samples depending on implementation of the system 100), trigger selection by the system 100 of a subset of features of the generated features, generate machine learning models (e.g., base models), initiate generation of an ensemble machine learning model (e.g., a combination of base models and/or permutations of the base models having the highest area under the receiver operating characteristic can be selected as the ensemble meta-learner/machine learning model). In certain embodiments, the first user 101 may be able to pause or stop operation of the system 100. In embodiments, the first user 101 can upload training data for training the models, such as via the first user device 102 and/or any other device of the system 100.

The system 100 may also include a communications network 135. The communications network 135 may be under the control of a service provider, any designated user, a computer, another network, or a combination thereof. The communications network 135 of the system 100 may be configured to link each of the devices in the system 100 to one another. For example, the communications network 135 may be utilized by the first user device 102 to connect with other devices within or outside communications network 135. Additionally, the communications network 135 may be configured to transmit, generate, and receive any information and data traversing the system 100. In certain embodiments, the communications network 135 may include any number of servers, databases, or other componentry. The communications network 135 may also include and be connected to a mesh network, a local network, a cloud-computing network, an IMS network, a VoIP network, a security network, a VOLTE network, a wireless network, an Ethernet network, a satellite network, a broadband network, a cellular network, a private network, a cable network, the Internet, an internet protocol network, MPLS network, a content distribution network, any network, or any combination thereof. Illustratively, servers 140, 145, and 150 are shown as being included within communications network 135. In certain embodiments, the communications network 135 may be part of a single autonomous system that is located in a particular geographic region or be part of multiple autonomous systems that span several geographic regions.

Notably, the functionality of the system 100 may be supported and executed by using any combination of the servers 140, 145, 150, and 160. The servers 140, 145, and 150 may reside in communications network 135, however, in certain embodiments, the servers 140, 145, 150 may reside outside communications network 135. The servers 140, 145, and 150 may provide and serve as a server service that performs the various operations and functions provided by the system 100. In certain embodiments, the server 140 may include a memory 141 that includes instructions, and a processor 142 that executes the instructions from the memory 141 to perform various operations that are performed by the server 140. The processor 142 may be hardware, software, or a combination thereof. Similarly, the server 145 may include a memory 146 that includes instructions, and a processor 147 that executes the instructions from the memory 146 to perform the various operations that are performed by the server 145. Furthermore, the server 150 may include a memory 151 that includes instructions, and a processor 152 that executes the instructions from the memory 151 to perform the various operations that are performed by the server 150. In certain embodiments, the servers 140, 145, 150, and 160 may be network servers, routers, gateways, switches, media distribution hubs, signal transfer points, service control points, service switching points, firewalls, routers, edge devices, nodes, computers, mobile devices, or any other suitable computing device, or any combination thereof. In certain embodiments, the servers 140, 145, 150 may be communicatively linked to the communications network 135, any network, any device in the system 100, or any combination thereof.

The database 155 of the system 100 may be utilized to store and relay information that traverses the system 100, cache content that traverses the system 100, store data about each of the devices in the system 100 and perform any other typical functions of a database. In certain embodiments, the database 155 may be connected to or reside within the communications network 135, any other network, or a combination thereof. In certain embodiments, the database 155 may serve as a central repository for any information associated with any of the devices and information associated with the system 100. Furthermore, the database 155 may include a processor and memory or may be connected to a processor and memory to perform the various operation associated with the database 155. In certain embodiments, the database 155 may be connected to the servers 140, 145, 150, 160, the first user device 102, the second user device 111, the additional user devices, any devices in the system 100, any process of the system 100, any program of the system 100, any other device, any network, or any combination thereof.

The database 155 may also store information and metadata obtained from the system 100, store metadata and other information associated with the first and second users 101, 110, store artificial intelligence/machine learning models (e.g., base models and ensemble models) utilized in the system 100, store sensor data, store samples (e.g., samples for molecules that are permeable, samples for molecules that are non-permeable, any other types of samples, or a combination thereof), store Simplified Molecular Input Line Entry System (SMILE) structures of a molecule, store translations or conversions of the SMILE structures, store features generated from the various samples, store fingerprint representations of molecules, store results of Chi-square tests conducted on the various features, store augmented samples (e g., such as when augmenting an imbalanced dataset), store information identifying which subset of features from the generated features have been selected by the system 100, store predictions made by the system 100 and/or artificial intelligence models, store information and/or content utilized to train the artificial intelligence models, store user profiles associated with the first and second users 101, 110, store device profiles associated with any device in the system 100, store communications traversing the system 100, store user preferences, store information associated with any device or signal in the system 100, store information relating to patterns of usage relating to the user devices 102, 111, store any information obtained from any of the networks in the system 100, store historical data associated with the first and second users 101, 110, store device characteristics, store information relating to any devices associated with the first and second users 101, 110, store information associated with the communications network 135, store any information generated and/or processed by the system 100, store any of the information disclosed for any of the operations and functions disclosed for the system 100 herewith, store any information generating by and/or traversing the system 100, or any combination thereof. Furthermore, the database 155 may be configured to process queries sent to it by any device in the system 100.

Notably, as shown in FIG. 1, the system 100 may perform any of the operative functionality disclosed herein by utilizing the processing capabilities of server 160, the storage capacity of the database 155, or any other component of the system 100 to perform the operative functions disclosed herein. The server 160 may include one or more processors 162 that may be configured to process any of the various functions of the system 100. The processors 162 may be software, hardware, or a combination of hardware and software. Additionally, the server 160 may also include a memory 161, which stores instructions that the processors 162 may execute to perform various operations of the system 100. For example, the server 160 may assist in processing loads handled by the various devices in the system 100, such as, but not limited to, obtaining samples for training one or more machine learning models (e.g., samples of data including information indicating whether a particular molecule is permeable or non-permeable, unlabeled sample data including information including characteristics, structural information, samples associated with prior predictions made by a machine learning model, samples indicating the accuracy of prior predictions made by a machine learning model, and/or other information associated with molecules and/or information associated with blood-brain barrier permeability); generating structural representations of molecules (e.g., SMILE representations), generating any number of features from the structural representations of the molecules, executing Chi-square tests (or other tests) on the features to determine whether blood-brain barrier permeability is dependent on fingerprint representations of the molecules; determining ratios of permeable and non-permeable samples associated with a molecule(s); augmenting the training dataset, such as when impermeable and permeable samples are imbalanced; reducing the amount of features utilized for the balanced training data set, such as by utilizing a technique such logistic regression to created a selected set of features for the balanced training dataset; training machine learning models to predict blood-brain barrier permeability; selecting candidate molecules for evaluation by a machine learning model; analyzing the candidate molecule and information associated with the candidate molecule (e.g., structural representations, fingerprint representations, etc.); generating predictions regarding the blood-brain barrier permeability of the candidate molecule; determining an accuracy of the prediction, such as by comparing the prediction to observable results associated with using the candidate molecule on a user (e.g., first user 101 or second user 110); retraining the machine learning models based on the comparison and based on new and/or updated sources of data; and performing any other operations conducted in the system 100 or otherwise. In certain embodiments, multiple servers 160 may be utilized to process the functions of the system 100. In certain embodiments, the server 160 and other devices in the system 100, may utilize the database 155 for storing data about the devices in the system 100 or any other information that is associated with the system 100. In certain embodiments, multiple databases 155 may be utilized to store data in the system 100.

Although FIGS. 1-9 illustrate specific example configurations of the various components of the system 100, the system 100 may include any configuration of the components, which may include using a greater or lesser number of the components. For example, the system 100 is illustratively shown as including a first user device 102, a second user device 111, a communications network 135, a server 140, a server 145, a server 150, a server 160, and a database 155. However, the system 100 may include multiple first user devices 102, multiple second user devices 111, multiple communications networks 135, multiple servers 140, multiple servers 145, multiple servers 150, multiple servers 160, multiple databases 155, or any number of any of the other components inside or outside the system 100. Furthermore, in certain embodiments, substantial portions of the functionality and operations of the system 100 may be performed by other networks and systems that may be connected to system 100.

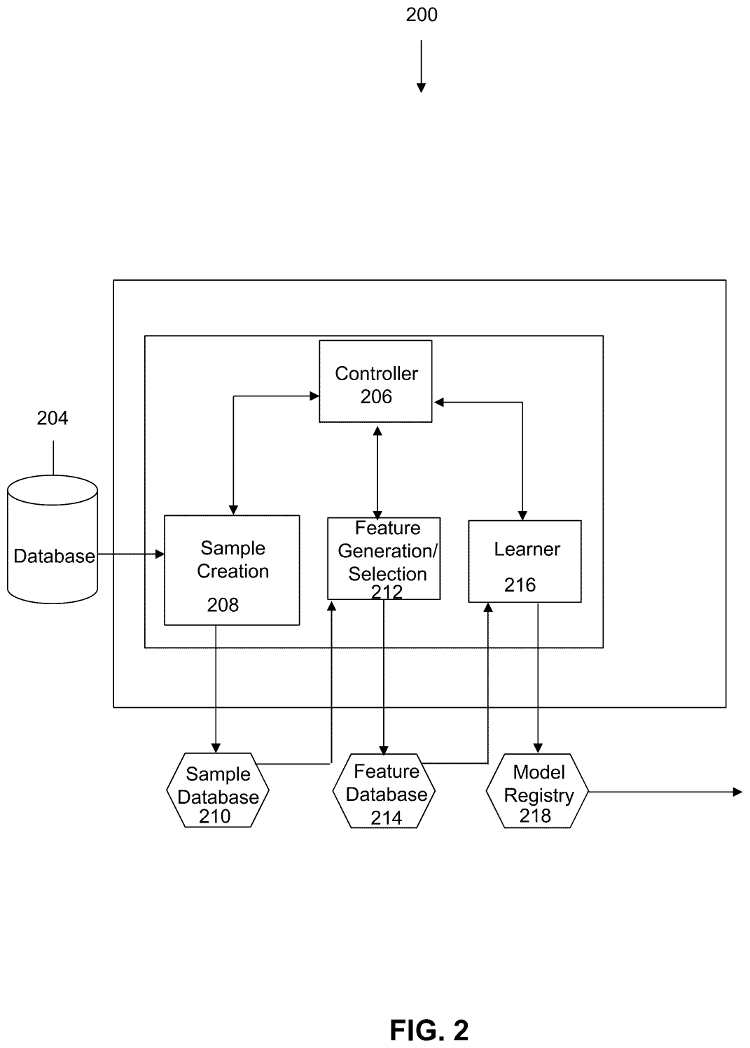

Referring now also to FIG. 2, an exemplary schematic diagram of a system 200 for building and training a machine learning model for predicting blood-brain barrier permeability is provided. In certain embodiments, the system 200 can be a part of system 100 and/or be connected to system 100. In certain embodiments, the functionality and components of system 100 can be combined with the functionality and components of system 200. In certain embodiments, the system 200 can include any number of components, such as, but not limited to, a database 204, a controller 206, a sample creation component 208 (e.g., module or software process), a sample database 210, a feature generation/selection component 212, a feature database 214, a learner 216 (e.g., base learner and/or ensemble meta-learner machine learning model), a model registry 218, any other components or a combination thereof. In certain embodiments, the database 204 can include data (e.g., raw data) obtained from a variety of data sources, such as, but not limited to, cloud computing systems, remote and/or local devices, applications, other database, or a combination thereof. The data can be data associated with any number of molecules, such as, but not limited to, information describing the molecule, information indicating characteristics of the molecule, information indicating the capabilities of the molecule, information indicating the chemical structure of the molecule, the permeability of the molecule (e.g., permeable, partially permeable, non-permeable, etc), any other information or a combination thereof.

In certain embodiments, the sample creation component 208 can generate samples from the raw data from the database 204 and can store the samples in the sample database 210 for future retrieval and/or use. Once the samples are generated, the feature generation/selection component 212 can extract features from the various samples stored in the sample database 210. The extracted features can be stored into a feature database 214 for future retrieval and/or use. In certain embodiments, the learner 216 (e g., base learner and/or ensemble meta-learner) can be trained using the features from the feature database 214 to generate predictions regarding the blood-brain barrier permeability of molecules. The models can be trained using the training data from the feature database 214, performance can be validated using a validation set of data, and testing using testing data. The finalized model can be stored in a model registry 218 and can be called upon by a software process to generate predictions, such as for a candidate molecule for which blood-brain barrier permeability predictions are desired. The models generated by the system 200 can be tuned and updated over time as new data, predictions, and/or the accuracy of the predictions is measured over time.

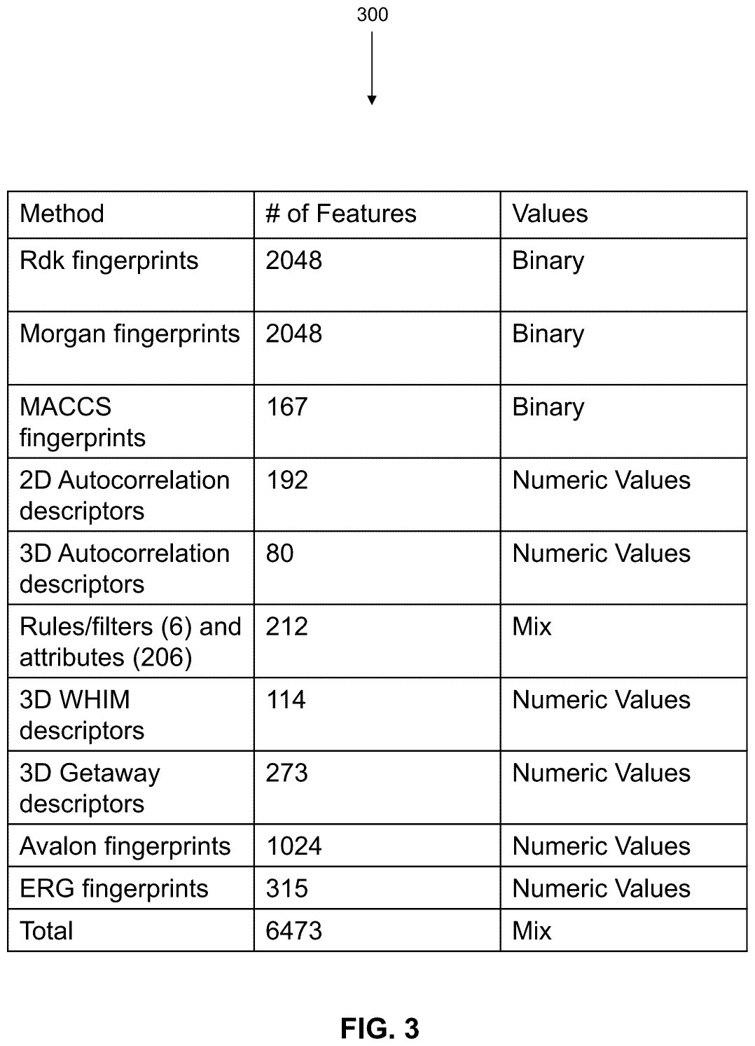

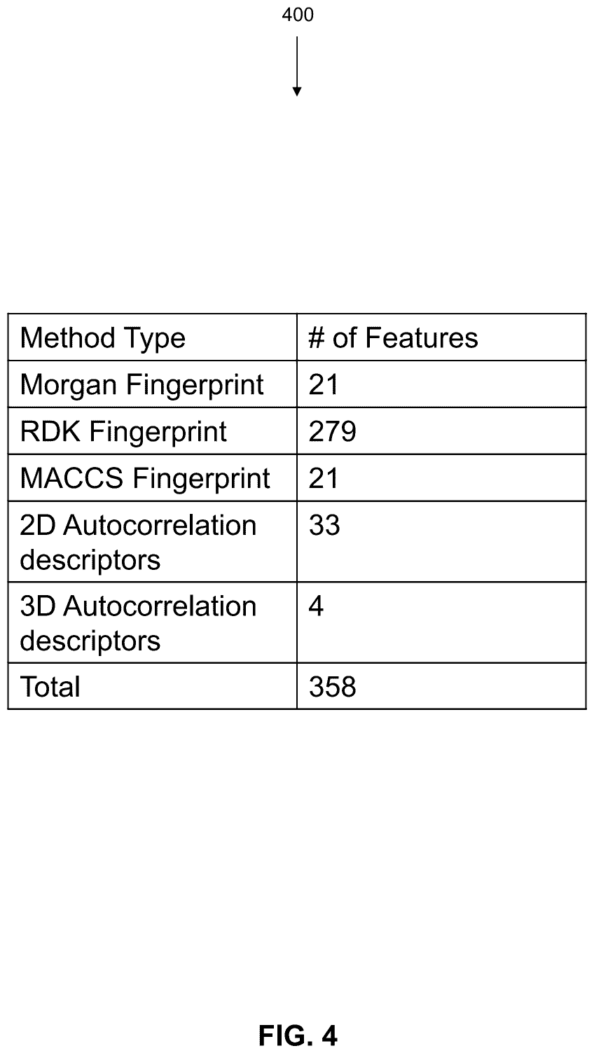

Operatively, the systems 100, 200 may operate and/or execute the functionality as described and illustrated in FIGS. 1-9 or as otherwise described herein. In certain embodiments, the system can select resulting features for machine learning and using regularized feature selection techniques in order to reduce the total number of features generated from a sample set into the most important features to save on computational resources and more effectively train machine learning models to perform predictions relating to blood-brain barrier permeability. In certain embodiments, such a process can be utilized to remove noise from the data by reducing the features. In certain embodiments, method utilized can be a penalty or bias-based regularized method, which can assign the different coefficients to each feature based on its weight in predicting blood-brain barrier permeability. Referring to FIG. 3, an exemplary table 300 illustrating 6473 features obtained from samples is shown, with an itemization of the number of samples for each category (e.g., Rdk fingerprints, Morgan fingerprints, MACCS fingerprints, Avalon fingerprints, ERG fingerprints, 2D autocorrelation descriptors, 3d autocorrelation descriptors, rules/filters and corresponding attributes, 3D WHIM descriptors, 3D Getaway descriptors, Avalon fingerprints, ERG fingerprints, etc.). As an example, 6,473 features extracted from samples can be reduced to 358 features, in order to use them for effective machine learning. Referring now also to FIG. 4, an exemplary table 400 illustrating a reduced set of 358 features is shown. In certain embodiments, when a molecular fingerprint representation of a molecule is generated and/or acquired, the similarity between the molecular fingerprint and another molecular fingerprint can be analyzed effectively.

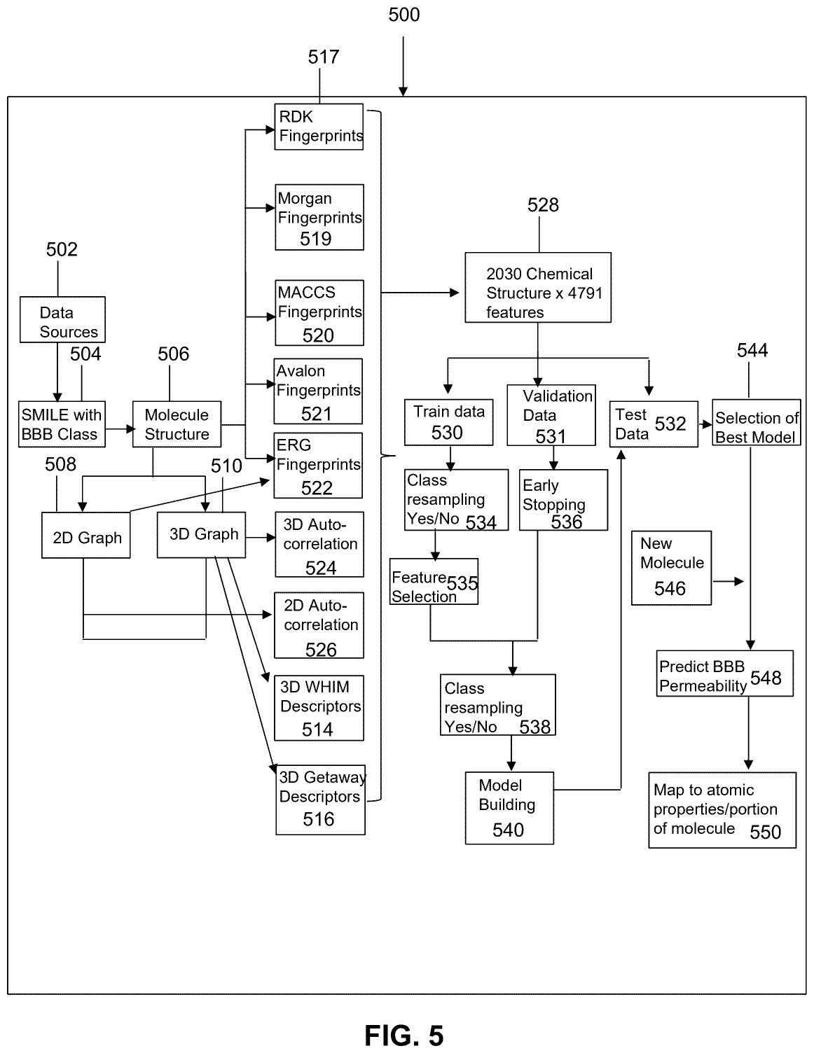

Referring now also to FIG. 5, an exemplary process flow diagram for a process flow 500 for generating features, reducing features, training a machine learning model, and analyzing a candidate molecule for blood-brain barrier permeability according to embodiments of the present disclosure is shown. The process flow 500 can be implemented by utilizing the system 100, the system 200, or a combination thereof. At 502, the process flow 500 can include obtaining data from various data sources 502 including, but not limited to Therapeutics Data Commons (TDC) data, data obtained via API calls to systems and/or applications, data obtained by other systems, data generated by the system 100 and/or system 200 itself, or a combination thereof. In certain embodiments, the data can be raw data, samples (e.g., labeled data (e.g., data associated with molecules that are labeled as permeable or non-permeable, chemical structure information for molecules, any other information associated with molecules, etc.) and/or unlabeled data) and/or other types of data. In certain embodiments, at 504, the process flow 500 can include obtaining and/or generating a structural representation, such as for molecules in the data obtained at 502. The structural representation can be a SMILE structure (i.e., chemical structure) from which features can be generated that can be utilized for model training. In certain embodiments, the features can include, but are not limited to, molecular fingerprint representations, descriptors (e g., feature descriptors, image descriptors, text descriptors, temporal descriptors, graph descriptors, etc. that include attributes or characteristics associated with the molecules in the data), embeddings, and/or any other types of features. In certain embodiments, the structural representations can also include an identification of the class of the sample, such as permeable or non-permeable (or semi-permeable).

At 506, the molecule's structure can be converted into a string of symbols that is interpretable and understood by software of the systems 100, 200. At 508, 510, 514, 516, 517, 519, 520, 521, 522, 524, 526 various types of features can be generated and/or extracted from the representations. For example, at 508, 2D graphs can be generated from the representations, at 510, 3D graphs can be generated from the representations. In certain embodiments, embeddings can be generated from the 2D and 3D graphs. In certain embodiments, for example, 2D and 3D descriptors can be generated from the embeddings, and, 3D descriptors can be generated from the embeddings. In certain embodiments, 2D autocorrelation features can be generated at 526 and 3D autocorrelation features can be generated at 524 (i.e., autocorrelation can involve computing the correlation between a pixel and neighboring pixels and/or in 3D volumes in various directions to enable identification of patterns in the data by a machine learning model). In certain embodiments, 3D WHIM descriptors 514 and 3D Getaway descriptors 516 can also be generated as well. In certain embodiments, 3D WHIM descriptors 514 can be 3D structural descriptors obtained from atomic coordinates of a 3D molecular structural representation of molecule, and can include information relating to the size, shape, symmetry, atom distribution, and/or other information associated with the molecule. In certain embodiments, the 3D Getaway descriptors (e.g., geometry, topology, and atom-weights assembly) 516 can be molecular descriptors that match 3D molecular geometry provided by a molecular influence matrix and atom relatedness by topology, and may include information, such as, but not limited to, atomic weights (e.g., atomic mass, polarizability, van der Waals volume, electronegativity, etc.). From the structural representations, various fingerprints can be generated, such as at, 517, 519, and 520, 521, and 522.

For example, RDK fingerprints 517 can be molecular fingerprints used to represent molecular structures in a format capable of being understood by the system 100 and for the machine learning models of the system 100. In certain embodiments, the RDK fingerprints 517 can be circular fingerprints that consider the circular neighborhood around each atom in the molecule. In certain embodiments, the fingerprints can be represented as a bit vector where each bit corresponds to the presence or absence of a substructure or pattern in the molecule. The RDK fingerprints 517 can also indicate the size of the fingerprint (e.g., length of bit vector). Morgan fingerprints can be utilized to represent the features of the molecule in a condensed format, such that they are suitable for processing by the system 100, 200. As with the RDK fingerprint 517, the Morgan fingerprint 519 can include information on the chemical environment around each atom in the molecule. Additionally, the Morgan fingerprint 519 can include encoding of substructures into the fingerprint (e.g., bond types, atom types, and/or connectivity patterns), hashing of substructures into a bit vector representation, the size of the fingerprint, and/or any other Morgan fingerprint 519 information. The Molecular Access System (MACCS) fingerprint 520 can be a binary fingerprint representing molecular structures as strings of binary bits. In certain embodiments, each bit represents the presence or absence of a specific substructure or molecular pattern. In certain embodiments, the MACCS fingerprint 520 can include fixed-length representation and predefined structural keys that indicate structural features of the molecules, such as, but not limited to, functional groups, ring systems, and/or other characteristic fragments. In certain embodiments, Avalon fingerprints 521 can also be generated. In certain embodiments, Avalon fingerprints 521 can represent chemical compounds associated with a molecule and can indicate chemical substructures or fragments within a molecule. In certain embodiments, the Avalon fingerprints 521 can encode global and local structural features of a molecule and can include information, such as, but not limited to, atom types, ring systems, bond information, and/or other molecular structure characteristics. In certain embodiments, the ERG fingerprints (e.g., extended reduced graphs) 522 can be based on graphs and can capture structural information in binary format (or another desired format). In certain embodiments, the ERG fingerprints 522 can include information on substructures within a radius around each atom in a molecule and the substructures can be hashed into a fixed-length bit strings, which can be used to generate a binary fingerprint that represents the molecule's structures features as indicated by the substructures.

Once the various features (e.g., fingerprint representations, descriptors, autocorrelations, etc.) are aggregated at 528, the process flow 500 can proceed to splitting the feature data at 530 to training data, at 531 to validation data, and at 532 to test data, which can be utilized in training, validation, and testing of generated machine learning models respectively by the systems 100, 200. In certain embodiments, the data can be prepared with various different seeds, which can involve utilizing slightly different molecule compositions for independent training and testing of models. Using the different seed options, the data records can be shuffled in each split. Such shuffling can facilitate averaging of the performance metrics and calculating the standard deviation. At 534, the systems 100, 200 can analyze the training data and determine whether the training data needs to be balanced, such as if the samples are imbalanced towards permeable samples versus non-permeable samples. Balancing/class resampling can be conducted by generating synthetic data for the minority class (i.e. the type of sample with fewer samples than the other type of sample) to balance out the permeable and non-permeable samples. At 535, feature selection can be conducted to select features that are important (e.g., features known to be important and are tagged by the systems 100, 200 as such, features that are shown and/or known to have correlation with blood-brain barrier permeability, etc.)

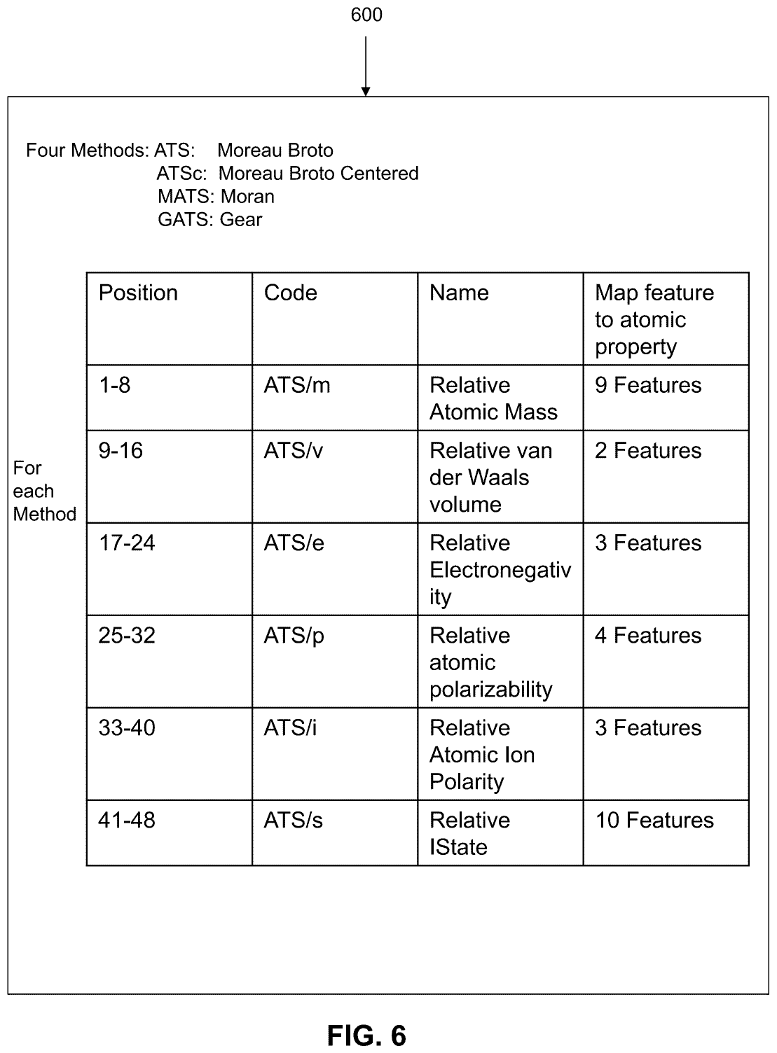

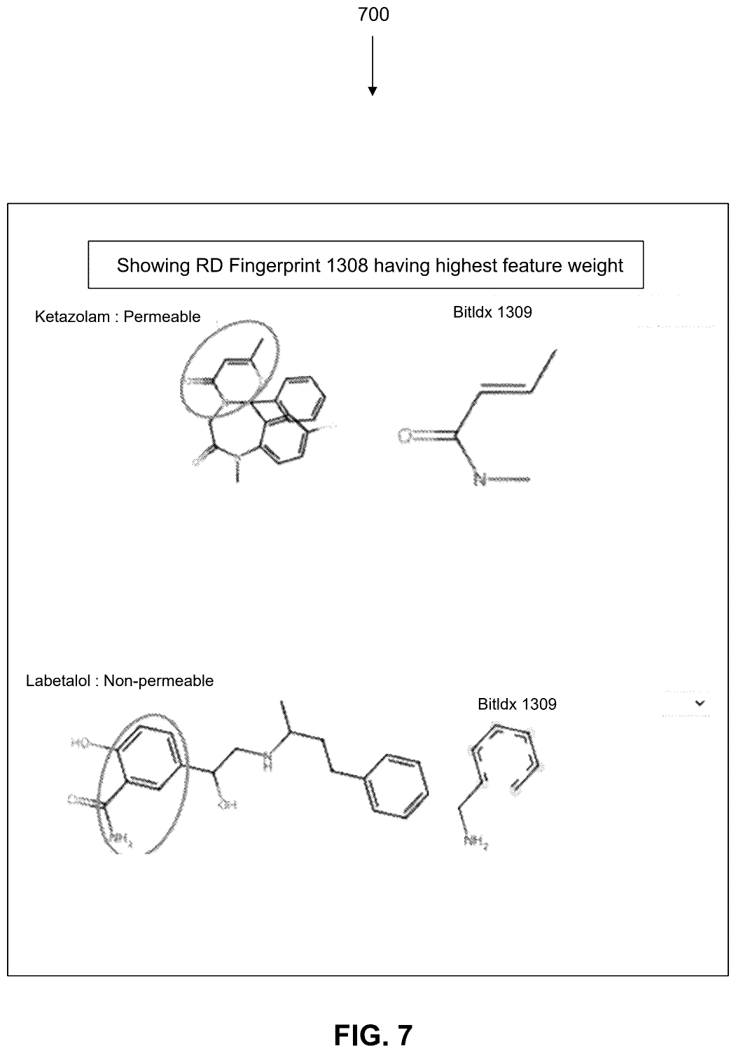

At 538, after feature selection is conducted, the process flow 500 can include conducting further class resampling if needed. At 540, one or more base learner models and/or ensemble learning models (described in the present disclosure) can be trained and/or built using the training data with the selected subset of features. At 531, validation data can be utilized to validate the performance of the generated and/or trained machine learning model(s). In certain embodiments, early stopping 536 can be conducted to prevent overfitting of the machine learning models during the training process. In certain embodiments, the early stopping 536 can include monitoring the performance of the machine learning model(s) via the validation dataset and stopping training when the performance of the model begins to degrade, instead of waiting for the model to complete all epochs or iterations. After early stopping is conducted at 536, the process flow 500 can include conducting class resampling 538, which can be utilized to train and validate the model(s). At 532, the machine learning model can be tested with test data to confirm the performance and predictive capability of the model(s) in determining bloodbrain barrier permeability of a molecule. At 544, the systems 100, 200 can select the optimal model (i.e., the model with the highest predictive performance, best use of computer resources, etc.) and the select, at 546, a candidate molecule to evaluate. The machine learning model(s) can analyze the candidate molecule and predict the molecule's blood-brain barrier permeability based on the training at 548. At 550, the machine learning model can map the top features (e.g., features having correlation to blood-brain barrier permeability) to atomic properties and/or structures/portions of the molecule, as shown in FIG. 6. In certain exemplary test scenarios, accuracy >0.912 and AUC of >0.928 on the test dataset was achieved and annotations of the 358 features to individual methods are shown in FIG. 4. Additionally, blood-brain barrier permeability with accuracy was shown in the test. This information can be utilized by the systems 100, 200 to understand and change the molecule to make the molecule blood-brain barrier permeable or non-permeable. In certain embodiments, the top features (e.g., High Shapley Additive explanations (SHAP) value) can be mapped back to atomic properties/portion of molecule, and the atomic relative state and relative atomic mass can have a role in deciding blood-brain barrier permeability, which belongs to the 2D autocorrelation method (e.g., as shown in FIG. 6). In certain scenarios, the chemical function groups can be matched with those correlating with blood-brain barrier permeability. In certain embodiments, the fingerprints/features can be mapped back to the portion of the molecule structure. An example is provided in FIG. 7, which shows the mapping of permeability or non-permeability to a specific portion of a molecule. In certain scenarios, while use of the deep neural network algorithm tends to overfit easily, the way the systems 100, 200 combine the feature selection and use validation data for early stopping is unique to derive a model with high accuracy and capability to identify blood-brain barrier permeability related important features.

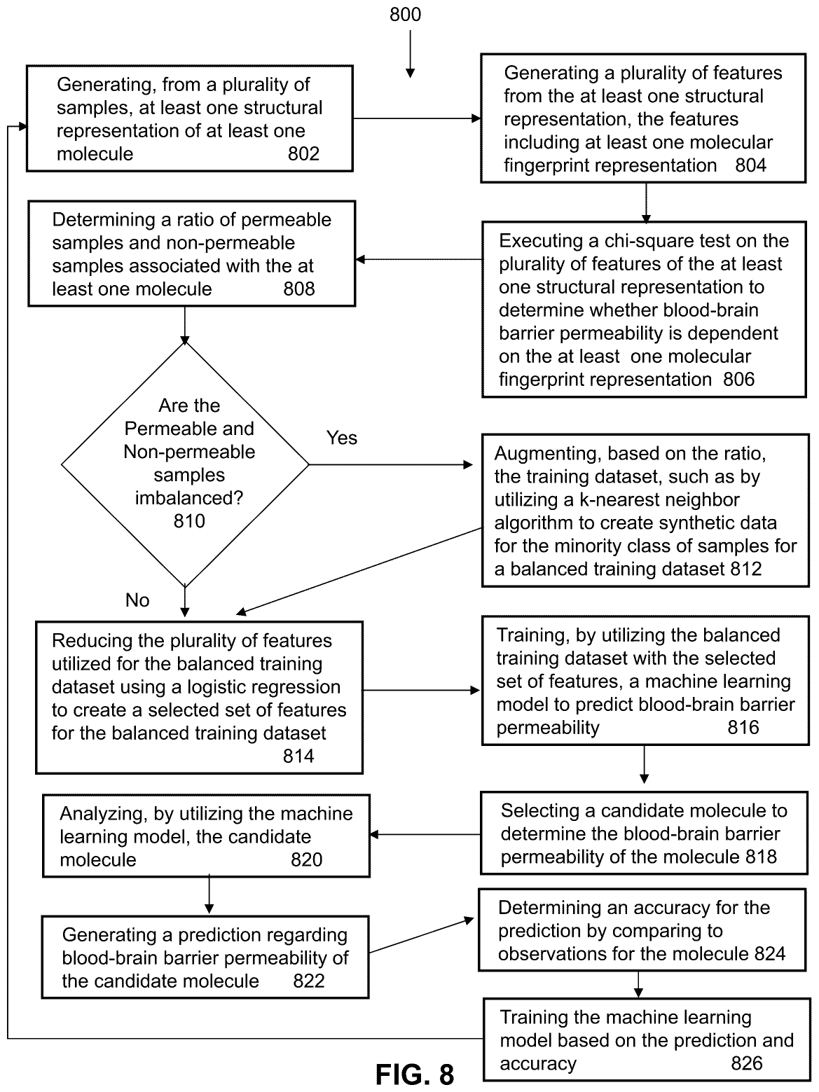

Notably, the system 100 may execute and/or conduct the functionality as described in the method(s) that follow. As shown in FIG. 8, an exemplary method 800 for building and utilizing a machine learning model for generating predictions regarding blood-brain barrier permeability for molecules, drugs, chemicals, or a combination thereof, is schematically illustrated. In certain embodiments, the method of FIG. 8 can be implemented in the systems of FIGS. 1-9 and/or any of the other systems, devices, and/or componentry illustrated in the Figures. In certain embodiments, the method of FIG. 8 may be performed by processing logic that can include hardware (e.g., processing device, circuitry, dedicated logic, programmable logic, microcode, hardware of a device, integrated circuit, etc), software (e.g., instructions run or executed on a processing device), or a combination thereof. In some embodiments, the method of FIG. 8 may be performed at least in part by one or more processing devices (e.g., processor 102, processor 122, processor 141, processor 146, processor 151, and processor 161 of FIG. 1). Although shown in a particular sequence or order, unless otherwise specified, the order of the steps in the method 800 may be modified and/or changed depending on implementation and objectives. Thus, the illustrated embodiments should be understood only as examples, and the illustrated processes can be performed in a different order, and some processes can be performed in parallel. Additionally, one or more processes can be omitted in various embodiments. Thus, not all processes are required in every embodiment. Other process flows are possible.

In certain embodiments, the method 800 and/or functionality and features supporting the method 800 may be conducted via an application of the system 100, machine learning and/or artificial intelligence models of the system 100, devices of the system 100, processes of the system 100, any component of the system 100, or a combination thereof. Generally, the method 800 may include steps for obtaining samples of data associated with molecules from various data sources, converting the samples into structural representations, and generating a plurality of features from the structural representations, such as fingerprint representations, descriptors, graph embeddings, and/or other representations. The method includes executing tests on the features to determine blood-brain barrier permeability dependency, such as based on a particular fingerprint representation of the molecule. The method includes analyzing the ratio of permeable to non-permeable samples in the samples of data and augments the samples with synthetic data to create a balanced dataset if an imbalance between the types of samples is determined by the system. The method further includes reducing the features utilized for training the machine learning utilizing techniques, such as logistic regression, to create a selected set of features for the balanced dataset. The method includes training a machine learning model using the balanced dataset and then utilizing the machine learning model to predict blood-brain barrier permeability for a candidate molecule.

At step 802, the method 800 may include generating and/or obtaining, from a plurality of samples, a structural representation(s) of one or more molecules. In certain embodiments, the samples can be labeled samples (e.g., labeled as permeable or as non-permeable or have other labels associated with a molecule) or unlabeled (e.g., such as when utilizing reinforcement learning with the system) In certain embodiments, the samples can include data and information obtained from a variety of data sources, such as, but not limited to, Therapeutics Data Commons data having SMILE chemical structures, textual chemical structures, visual chemical structures, sound descriptions of chemical structures, audiovisual chemical structures, and/or any other types of structures for any number and/or type of molecules, such as chemical molecules. In certain embodiments, the SMILE structure can represent a particular molecule's structure and can comprise a string of characters (e.g., ASCII) that uniquely represent the structure of the molecule. In certain embodiments, the structure can include stereochemistry and/or connectivity information and can be configured to be human-readable, machine-readable, or a combination thereof. In certain embodiments, the structure can include atomic symbols (e.g., O for oxygen), bond symbols (e.g., single bond, double bond ‘=’, triple bond etc.), isotope information, chirality (e.g., using ‘@’ and 7′ to represent chiral centers and cis-trans isomerism), hydrogen information (e.g., appending H to the representation for explicit hydrogens), branching and ring structures, and/or any other information. In certain embodiments, the generating and/or obtaining of the structural representations from the samples may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. In certain embodiments, the samples themselves can be obtained from data sources, such as, but not limited to, data repositories, databases, cloud systems and/or networks, live data feeds, API calls to third-party systems, any other data sources, or a combination thereof.

At step 804, the method 800 can include generating a plurality of features from the one or more structural representations. In certain embodiments, the features can be numerical and/or other types of features and can include one or more fingerprint representations (e.g., Morgan fingerprints, RDKit (RDK) fingerprints, MACCS fingerprints, Avalon fingerprints, ERG fingerprints, and/or other types of fingerprints, descriptors (e.g., 2D and/or 3D autocorrelation descriptors, 3D WHIM descriptors, and/or 3D Getaway descriptors), graph embeddings (e.g., graph embeddings based on 2D and/or 3D structures), and/or any other types of features. In certain embodiments, numerical features (e.g., some can be binary and some can be non-binary) can be considered as a proxy to different atomic properties, such as connectivity (element, number of heavy neighbors, number of hydrogens (Hs), charge, isotope), chemical features (e.g., donor, acceptor, aromatic, halogen, basic, acidic, etc.), bond type, atomic mass, electrotolpological states. In certain embodiments, additional features such as atomic rules (e.g. Lipinski rules, Ghose filter, Veber filter etc.) and their corresponding attributes can be utilized as well. The features can be utilized to feed a machine learning model for training purposes, such as to build and train a machine learning model to perform blood-brain barrier permeability predictions for molecules of interest. In certain embodiments, the generating of the plurality of features may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 806, the method 800 can include performing one or more tests to determine whether permeability is dependent on a particular fingerprint (and/or other feature) as part of feature engineering according to the method 800. In certain embodiments, for example, a Chi-square statistical test can be performed for all features (e.g., individual fingerprint features) to determine whether permeability is dependent on the fingerprint (and/or other feature). In certain embodiments, fingerprints with a p-value less than 0.05 can be considered as significant in a first phase. In certain embodiments, in a second phase, the ratio of permeable and non-permeable samples can be calculated for drug molecule samples containing each fingerprint. In certain embodiments, fingerprints with a permeability of 50% or lower can be categorized as significant negatively-associated fingerprints, and the fingerprints with 80% or greater permeability can be considered significant positively-associated fingerprints. In certain embodiments, the threshold for negative or positive association can be adjusted to desired values. In certain embodiments, new features are created totaling the count of negatively and positively-associated fingerprints in each drug molecule sample. In certain embodiments, the one or more tests may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 808, the method 800 can include determining a ratio of samples (e.g., drug molecule samples) that are permeable to the samples that are non-permeable. In certain embodiments, the samples can include training samples, validation samples, and testing samples. Often times, there is an imbalance in the samples that are obtained from data sources. For example, in certain datasets there may be significantly more permeable samples versus non-permeable samples. In certain embodiments, the determining of the ratio can be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. At step 810, the method 800 can include determining if the permeable and non-permeable samples are imbalanced, such as if the samples are not equal or are not within a threshold number of samples of the other. In certain embodiments, the determining may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

If there is an imbalance, the method 800 can proceed to step 812. At step 812, which can include augmenting the dataset (e.g., the training dataset) to correct the imbalance in the samples. For example, various techniques can be utilized to augment the dataset, such as utilizing a k-nearest neighbor algorithm on the samples to create synthetic data for the minority class (i.e., the type of sample with fewer samples than the other type of sample) for provide a balanced training dataset. For example, in an exemplary scenario, due to the class imbalance between the majority of drug samples being permeable (75.3%) and minority of samples being non-permeable (24.7%), Synthetic Minority Oversampling Technique (SMOTE) can be performed on the training set of data utilizing a k-nearest neighbor algorithm to create synthetic data for the minority class (e.g., the type of sample having few samples than the other type of sample) until the sample counts are balanced between permeable and non-permeable observations. This augmentation on the training data set can serve as an input to feature selection and model training phases of the workflow relating to building the machine learning model to make the predictions for blood-brain barrier permeability. In certain embodiments, the augmented samples may only be generated for samples utilized for training a machine learning model and not for the validation or test sets of data samples. In certain embodiments, the augmenting of the dataset can be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

If, however, at step 810, there is no imbalance in the samples, the method 800 may proceed directly from step 810 to 814, or, in the event the augmenting of the dataset has been performed at step 812, the method 800 may proceed from step 812 to 814. At step 814, the method 800 may include reducing the plurality of features (e.g., generated features) utilized for the balanced training dataset using any number of techniques, such as for conducting feature selection from the total set of features. For example, the features can be reduced using a logistic regression with least absolute shrinkage and a selection operator penalty to shrink the least important feature's coefficients to zero, thereby eliminating from features selected for use in model training. In certain embodiments, ten-fold cross-validation may be performed on a search range for the LI regularization parameter value, which provides the highest average accuracy across the folds. The LI regularization value identified as optimal in the search may be used to train a final feature selection model that eliminates features with a coefficients of zero to be removed, and remaining features are ranked as most important by the absolute value of their coefficient. In certain embodiments, the feature selection process described above can be performed once for just the subset of fingerprint features and again considering all types of features generated. In certain embodiments, two separate feature selection lists can be used in separate models in a next phase to generate diversity in model training and predictions. In certain embodiments, the reducing of the features and/or features selection can be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 816, the method can include training, such as by utilizing the balanced training dataset with the selected set of features, one or more machine learning models to predict blood-brain barrier permeability. In certain embodiments, for example, any number of base learners can be trained, which can then ultimately be utilized to create and/or train an ensemble meta-learner machine learning model. In certain embodiments, various methods can be utilized for base learner design and training. The method can include modeling for base learners that are utilized to generate diverse methods of predictions that serve as inputs to the ensemble meta-learner in a subsequent phase. In certain embodiments, logistic regression can be utilized. In certain embodiments, during feature selection, LI regularization may have been utilized to reduce features. In certain embodiments, the base learner model may not include further regularization and may serve to provide an easily interpretable model based on univariate effects of each feature used in training. Another method of training and/or designing the base learner models can involve using deep neural networks. In certain embodiments, the design of the neural network may be generated using a search on the optimal architecture for a range of two to five fully connected dense hidden layers (or other desired number range). Each hidden dense layer can include L2 regularization and may be followed by a dropout layer. The search additionally includes a range of neurons to use in each hidden layer. Each iteration of the architecture search can include early stopping criteria using a holdout subset from the training data to stop model training at the epoch which represents the highest area under the receiver operating characteristic curve (AUC-ROC) on the holdout samples. In certain embodiments, a final model can be constructed with the optimal number of layers and neurons identified in the search and can be fully trained up to the early stopping criteria. In certain embodiments, each base learner model can be trained with the augmented training data for each set of feature lists identified during the feature selection phase. After training, the predicted probability of permeability can be calculated on holdout validation samples that were not included in the model's training samples. In certain embodiments, the validation sample predictions can be used in training the subsequent ensemble meta-learner.

In certain embodiments, the trained base learner models can be evaluated by utilizing the validation samples to evaluate the performance of the models so that the parameters (e g., hyperparameters and/or other parameters) for the models can be tuned and/or adjusted to enhance performance of the predictive capability for predicting blood-brain barrier permeability. Validation sample predictions from base learner models can be used as feature inputs to the logistic regression meta-learner ensemble model. In certain embodiments, all base models can be evaluated as meta-learner inputs and permutations of the base-learner combinations and the combination of base-learners with the highest area under the receiver operating characteristic curve can be selected as the final meta-learner that is utilized to perform the blood-brain barrier predictions on candidate molecules. In certain embodiments, once the validation set of samples has been utilized to evaluate and tune performance of the models, such as on an iterative basis, the testing samples can be utilized to provide an unbiased evaluation of the model performance. In certain embodiments, generating predictions on the test set for evaluation is completed in two phases. First the probability of permeability for each sample can be predicted for each base learner and its associated selected features. Second, the test set base learner probabilities can be used as inputs to the meta-learner ensemble model for the final predictions.

In certain embodiments, the machine learning/artificial intelligence model may be, may include, and/or may utilize a Deep Convolutional Neural Network, a one-dimensional convolutional neural network, a two-dimensional convolutional neural network, a Long Short-Term Memory network, autoencoders, generative adversarial networks, vision transformers, any type of machine learning system, any type of artificial intelligence system, or a combination thereof. In certain embodiments, the models may incorporate the use of any type of artificial intelligence and/or machine learning algorithms to facilitate the operation of the artificial intelligence model(s). Notably, the system 100 may utilize any number of artificial intelligence models. The system 100 may train the artificial intelligence model(s) to reason and learn from data/information fed into the system 100 so that the model may generate and/or facilitate the generation of predictions about new data and information that is fed into the system 100 for analysis. As an example, the machine learning model(s) may be trained with data samples, such as, but not limited to, images, video content, audio content, text content, augmented reality content, virtual reality content, information relating to patterns, information relating to molecules, any type of data, or a combination thereof. The data that is utilized to train the artificial intelligence model may be utilized by the artificial intelligence model to predict whether a particular molecule is blood-brain barrier permeable.

Once the ensemble meta-learner is created from the base learner models, the method 800 can proceed to step 818. At step 818, the method 800 can include selecting a candidate molecule for evaluation. In certain embodiments, the selection of the candidate molecule can be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. At step 820, the method 800 may include analyzing, such as by utilizing the ensemble meta-learner machine learning model, the candidate molecule. In certain embodiments, the analyzing can be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 822, the method 80 can include generating a prediction regarding bloodbrain barrier permeability of the candidate. In certain embodiments, the prediction can be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. At step 824, the method 800 may include determining an accuracy for the prediction. In certain embodiments, for example, determining the accuracy can include comparing the prediction made by the machine learning model to observable results that result from a user (e.g., second user 110) using the molecule, such as during a treatment. In certain embodiments, the determination of the accuracy can be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. At step 826, the method 800 can include training the machine learning model (e.g., the ensemble meta-learner and/or base learners) based on the prediction made, the accuracy of the prediction, updated samples of data, new samples of data, or a combination thereof. The process can be repeated as desired so that the predictive capability of the machine learning model(s) improves over time. Notably, the method 800 may further incorporate any of the features and functionality described for the system 100, any other method disclosed herein, or as otherwise described herein.

The systems and methods disclosed herein may include still further functionality and features. For example, the operative functions of the system 100 and method may be configured to execute on a special-purpose processor specifically configured to carry out the operations provided by the system 100 and method. Notably, the operative features and functionality provided by the system 100 and method may increase the efficiency of computing devices that are being utilized to facilitate the functionality provided by the system 100 and the various methods discloses herein. For example, by training the system 100 over time based on data and/or other information provided and/or generated in the system 100, a reduced amount of computer operations may need to be performed by the devices in the system 100 using the processors and memories of the system 100 than compared to traditional methodologies. In such a context, less processing power needs to be utilized because the processors and memories do not need to be dedicated for processing. As a result, there are substantial savings in the usage of computer resources by utilizing the software, techniques, and algorithms provided in the present disclosure. In certain embodiments, various operative functionality of the system 100 may be configured to execute on one or more graphics processors and/or application specific integrated processors.

Notably, in certain embodiments, various functions and features of the system 100 and methods may operate without any human intervention and may be conducted entirely by computing devices. In certain embodiments, for example, numerous computing devices may interact with devices of the system 100 to provide the functionality supported by the system 100. Additionally, in certain embodiments, the computing devices of the system 100 may operate continuously and without human intervention to reduce the possibility of errors being introduced into the system 100. In certain embodiments, the system 100 and methods may also provide effective computing resource management by utilizing the features and functions described in the present disclosure. For example, in certain embodiments, devices in the system 100 may transmit signals indicating that only a specific quantity of computer processor resources (e.g. processor clock cycles, processor speed, etc.) may be devoted to training the artificial intelligence model(s), generating the features from the samples, executing tests on the features, rebalancing imbalanced data sample sets, reducing the features utilize to train the models, analyzing candidate molecules to predict blood-brain barrier permeability, determining the accuracy of the predictions, retraining the machine learning models, and/or performing any other operation conducted by the system 100, or any combination thereof. For example, the signal may indicate a number of processor cycles of a processor may be utilized to update and/or train an artificial intelligence model, and/or specify a selected amount of processing power that may be dedicated to generating or any of the operations performed by the system 100. In certain embodiments, a signal indicating the specific amount of computer processor resources or computer memory resources to be utilized for performing an operation of the system 100 may be transmitted from the first and/or second user devices 102, 111 to the various components of the system 100.

In certain embodiments, any device in the system 100 may transmit a signal to a memory device to cause the memory device to only dedicate a selected amount of memory resources to the various operations of the system 100. In certain embodiments, the system 100 and methods may also include transmitting signals to processors and memories to only perform the operative functions of the system 100 and methods at time periods when usage of processing resources and/or memory resources in the system 100 is at a selected value. In certain embodiments, the system 100 and methods may include transmitting signals to the memory devices utilized in the system 100, which indicate which specific sections of the memory should be utilized to store any of the data utilized or generated by the system 100. Notably, the signals transmitted to the processors and memories may be utilized to optimize the usage of computing resources while executing the operations conducted by the system 100. As a result, such functionality provides substantial operational efficiencies and improvements over existing technologies.

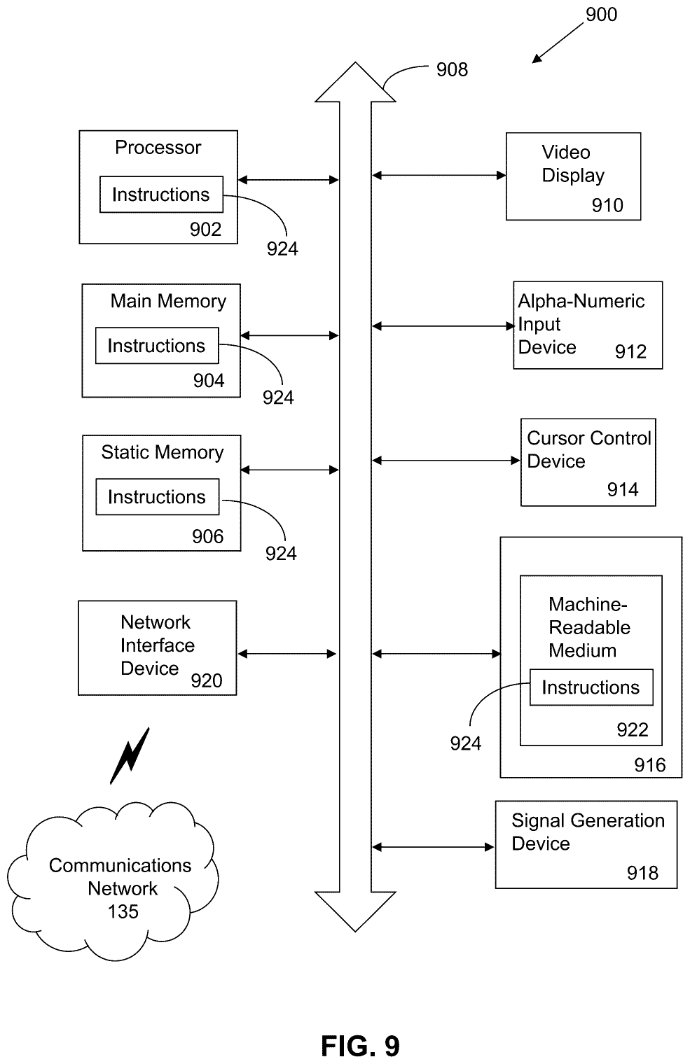

Referring now also to FIG. 9, at least a portion of the methodologies and techniques described with respect to the exemplary embodiments of the system 100 can incorporate a machine, such as, but not limited to, computer system 900, or other computing device within which a set of instructions, when executed, may cause the machine to perform any one or more of the methodologies or functions discussed above. The machine may be configured to facilitate various operations conducted by the system 100. For example, the machine may be configured to, but is not limited to, assist the system 100 by providing processing power to assist with processing loads experienced in the system 100, by providing storage capacity for storing instructions or data traversing the system 100, or by assisting with any other operations conducted by or within the system 100. As another example, the computer system 900 may assist with generating models associated with generating predictions relating blood-brain barrier permeability of a molecule, any type of predictions generated by the system 100, or a combination thereof. As another example, the computer system 600 may assist with training machine learning models of the system 100, selecting features for training the machine learning models of the system 100, generating fingerprint representations of molecules, identifying portions of a molecule associated with blood-brain barrier permeability, providing any other functionality provided by the system 100, or a combination thereof.

In certain embodiments, the machine may operate as a standalone device. In some embodiments, the machine may be connected (e.g., using communications network 135, another network, or a combination thereof) to and assist with operations performed by other machines and systems, such as, but not limited to, the first user device 102, the second user device 111, the server 140, the server 145, the server 150, the database 155, the server 160, any other system, program, and/or device, or any combination thereof. The machine may be connected with any component in the system 100. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 900 may include a processor 902 (e.g., a central processing unit (CPU), a graphics processing unit (GPU, or both), a main memory 904 and a static memory 906, which communicate with each other via a bus 908. The computer system 900 may further include a video display unit 910, which may be, but is not limited to, a liquid crystal display (LCD), a flat panel, a solid state display, or a cathode ray tube (CRT). The computer system 900 may include an input device 912, such as, but not limited to, a keyboard, a cursor control device 914, such as, but not limited to, a mouse, a disk drive unit 916, a signal generation device 918, such as, but not limited to, a speaker or remote control, and a network interface device 920.

In certain embodiments, the disk drive unit 916 may include a machine-readable medium 922 on which is stored one or more sets of instructions 924, such as, but not limited to, software embodying any one or more of the methodologies or functions described herein, including those methods illustrated above. The instructions 924 may also reside, completely or at least partially, within the main memory 904, the static memory 906, or within the processor 902, or a combination thereof, during execution thereof by the computer system 900. In certain embodiments, the main memory 904 and the processor 902 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 922 containing instructions 924 so that a device connected to the communications network 135, another network, or a combination thereof, can send or receive voice, video or data, and communicate over the communications network 135, another network, or a combination thereof, using the instructions. The instructions 924 may further be transmitted or received over the communications network 135, another network, or a combination thereof, via the network interface device 920.

While the machine-readable medium 922 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more readonly (non-volatile) memories, random access memories, or other re-writable (volatile) memories; magneto-optical or optical medium such as a disk or tape; or other self-contained information archive or set of archives is considered a distribution medium equivalent to a tangible storage medium. In certain embodiments, the “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure is not limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

We claim:

1. A system, comprising:
a memory that stores instructions; and
a processor configured to execute the instructions to configure the processor to:
generate a plurality of features for at least one structural representation of at least one molecule, wherein the plurality of features comprise at least one molecular fingerprint representation associated with the at least one molecule;
execute a chi-square test on the plurality of features of the at least one structural representation to determine whether blood-brain barrier permeability is dependent on the at least one molecular fingerprint representation;
determine a ratio of permeable samples and non-permeable samples associated with the at least one molecule and containing the at least one molecular fingerprint representation;
augment, based on the ratio, a training dataset comprising the permeable samples and non-permeable samples by utilizing a k-nearest neighbor algorithm to create synthetic data for a minority class of the permeable and non-permeable samples until sample counts for the training dataset are balanced between the permeable samples and non-permeable samples to generate a balanced training dataset;
reduce the plurality of features utilized for the balanced training dataset using a logistic regression with least absolute shrinkage to create a selected set of features for the balanced training dataset;
train, by utilizing the balanced training dataset with the selected set of features, an ensemble meta learner to predict blood-brain barrier permeability;
analyze, by utilizing the ensemble meta learner, a candidate molecule for bloodbrain barrier permeability; and
generate, by utilizing the ensemble meta learner, a prediction of whether the candidate molecule has blood-brain barrier permeability.

2. The system of claim 1, wherein the processor is further configured to generate the at least one structural representation of the at least one molecule by translating a three-dimensional structure of the at least one molecule into a string of symbols discernible by the system.

3. The system of claim 1, wherein the plurality of features further comprise descriptors, graph embeddings, or a combination thereof.

4. The system of claim 1, wherein the processor is further configured to determine that blood-brain barrier permeability is dependent on the at least one molecular fingerprint representation based on the at least one molecular fingerprint having a p-value of less than 0.05.

5. The system of claim 1, wherein the processor is further configured to classify the permeable samples of the plurality of samples as permeable based on fingerprints associated with the permeable samples having a threshold blood-brain permeability, and wherein the processor is further configured to classify the non-permeable samples of the plurality of samples as non-permeable based on fingerprints associated with the non-permeable samples having less than the threshold blood-brain permeability.

6. The system of claim 1, wherein the processor is further configured to determine that the non-permeable samples are the minority class in the plurality of samples based on the permeable samples being greater in number than the non-permeable samples.

7. The system of claim 1, wherein the processor is further configured to reduce, using the logistic regression, coefficients of features of the plurality of features to zero to eliminate the features from being included in the selected set of features.

8. The system of claim 1, wherein the processor is further configured to rank features in the selected set of features in order of importance based on an absolute value of each coefficient of the features in the selected set of features.

9. The system of claim 1, wherein the processor is further configured to generate the ensemble meta learner from at least one base learner model that is trained based on the balanced training dataset and by utilizing the logistic regression, a deep neural network, or a combination thereof.

10. The system of claim 1, wherein the processor is further configured to determine a predicted probability of permeability for holdout validation samples not included in the balanced training dataset.

11. The system of claim 10, wherein the processor is further configured to utilize the predicted probability of permeability for the holdout validation samples as an input to a logistic regression meta-learner ensemble model.

12. The system of claim 1, wherein the processor is further configured to select the ensemble meta learner as a combination of base learner models having a highest area under a receiver operating characteristic curve.

13. A method, comprising:
generating, by utilizing instructions from a memory that are executed by a processor, a plurality of features for at least one structural representation of at least one molecule, wherein the plurality of features comprise at least one molecular fingerprint representation associated with the at least one molecule;
executing, by utilizing the instructions from the memory that are executed by the processor, a chi-square test on the plurality of features of the at least one structural representation to determine whether blood-brain barrier permeability is dependent on the at least one molecular fingerprint representation;
determining a ratio of permeable samples and non-permeable samples associated with the at least one molecule and containing the at least one molecular fingerprint representation;
augmenting, based on the ratio, a training dataset comprising the permeable samples and non-permeable samples by utilizing a k-nearest neighbor algorithm to create synthetic data for a minority class of the permeable and non-permeable samples until sample counts for the training dataset are balanced between the permeable samples and non-permeable samples to generate a balanced training dataset;
reducing the plurality of features utilized for the balanced training dataset using a logistic regression with least absolute shrinkage to create a selected set of features for the balanced training dataset;
training, by utilizing the balanced training dataset with the selected set of features, an ensemble meta learner to predict blood-brain barrier permeability;
analyzing, by utilizing the ensemble meta learner, a candidate molecule for bloodbrain barrier permeability; and
generating, by utilizing the ensemble meta learner and by utilizing the instructions from the memory that are executed by the processor, a prediction of whether the candidate molecule has blood-brain barrier permeability.

14. The method of claim 13, further comprising identifying, by utilizing the ensemble meta learner, a specific portion of the candidate molecule that has the blood-brain barrier permeability.

15. The method of claim 13, further comprising generating the ensemble meta learner from at least one base learner model that is trained based on the balanced training dataset and by utilizing the logistic regression, a deep neural network, or a combination thereof.

16. The method of claim 13, further comprising stopping the training of at least one base learner model utilized to generate the ensemble meta learner at an epoch representing a highest area under a receiver operating characteristic curve on holdout samples.

17. The method of claim 13, further comprising reducing, using the logistic regression, coefficients of features of the plurality of features to zero to eliminate the features from being included in the selected set of features.

18. The method of claim 13, further comprising generating the at least one structural representation of the at least one molecule by translating a three-dimensional structure of the at least one molecule into a string of symbols.

19. The method of claim 13, further comprising determining a correlation of blood-brain permeability between the at least one molecule and the at least one candidate molecule.

20. A non-transitory computer-readable device comprising instructions, which, when loaded and executed by a processor, cause the processor to be configured to:
generate a plurality of features for at least one structural representation of at least one molecule, wherein the plurality of features comprise at least one molecular fingerprint representation associated with the at least one molecule;
execute a chi-square test on the plurality of features of the at least one structural representation to determine whether blood-brain barrier permeability is dependent on the at least one molecular fingerprint representation;
determine a ratio of permeable samples and non-permeable samples associated with the at least one molecule and containing the at least one molecular fingerprint representation;
augment, based on the ratio, a training dataset comprising the permeable samples and non-permeable samples by utilizing a k-nearest neighbor algorithm to create synthetic data for a minority class of the permeable and non-permeable samples until sample counts for the training dataset are balanced between the permeable samples and non-permeable samples to generate a balanced training dataset;
reduce the plurality of features utilized for the balanced training dataset using a logistic regression with least absolute shrinkage to create a selected set of features for the balanced training dataset;
train, by utilizing the balanced training dataset with the selected set of features, an ensemble meta learner to predict blood-brain barrier permeability;
analyze, by utilizing the ensemble meta learner, a candidate molecule for blood-brain barrier permeability; and
generate, by utilizing the ensemble meta learner, a prediction of whether the candidate molecule has blood-brain barrier permeability.
